(19) United States
(12) Patent Application Publication
(10) Pub. No.: US 2024/0216658 A1
(43) Pub. Date: Jul. 4, 2024

(54) BIODEGRADABLE SPINY MILLI-BALL ROBOT

(71) Applicant: The Hong Kong University of Science and Technology, Hong Kong (CN)

(72) Inventor: Yajing SHEN, Hong Kong (CN)

(21) Appl. No.: 18/394,726

(22) Filed: Dec. 22, 2023

Related U.S. Application Data
(60) Provisional application No. 63/478,497, filed on Jan. 4, 2023.

(51) Int. Cl. A61M 37/00

(52) U.S. Cl. CPC A61M 37/0015; A61M 2037/0023; A61M 2037/0053; A61M 2037/0061; A61M 2205/0238

(57) ABSTRACT

A drug administering device includes a spherical body, a magnetic actuator, and a plurality of microneedles. The magnetic actuator is arranged within the spherical body and configured to respond to an externally applied magnetic field. The plurality of microneedles are arranged on the spherical body, and the microneedles are configured to penetrate tissue.

### CROSS-REFERENCE TO RELATED APPLICATIONS

This patent application claims the benefit of U.S. Provisional Patent Application No. 63/478,497, filed on Jan. 4, 2023, which is hereby incorporated by reference.

### BACKGROUND

Oral drug administration is a preferred method for treating patients because it is non-invasive in nature, carries low risk of leading to infection, and is associated with a high rate of patient compliance. However, due to limited rapid degradation and poor absorption in the gastrointestinal (GI) tract, preclinical technologies for GI-based orally bioavailable biologic dosage forms cannot guarantee adequate bioavailability, which in some cases is only 1%. Moreover, macromolecular drugs (e.g., insulin, monoclonal antibodies, growth factor, recombined vaccine, etc.) are completely unabsorbable in GI tracts and may thus require subcutaneous injection to be employed in therapy, leading to patient inconvenience and discomfort. The drawbacks in high dose, low concentration, and poor oral administration bioavailability make oral drug administration undesirable for many promising therapy strategies, such as macromolecular drug-disease medication, selected high-dose chemotherapy, and local inflammation treatment.

Recent progress in intelligent miniature delivery systems offer new opportunities for conventional oral administration methods in controllability and functionality. Specifically, some physical delivery systems have demonstrated the ability to deliver macromolecular drugs in the stomach and intestine by ejection mechanisms, such as orally ingested self-orienting systems for insulin injection in the stomach, propelling devices with multiple drug-loaded microneedles for macromolecular drug injection in the intestine, and bioinspired mechanochemical therapeutic grippers that can release analgesic ketorolac tromethamine for anti-inflammatory therapy. However, despite these achievements, known needles or components for injection are usually non-degradable, as they are made of metals, plastics, or polymers. Such non-degradable materials are not ideal for digestion or elimination, and may cause immune rejection and intestinal obstruction. Moreover, due to the lack of active actuation ability, steerable locomotion in the GI tract and targeted release in specific localized areas remain as major challenges for these devices.

In comparison to known intelligent miniature delivery systems and devices, miniature robots have superior locomotion, a flexible nature, and untethered control ability. Accordingly, diverse types of small robots with high potential for use in targeted drug delivery inside the GI tract have been proposed. Such proposals include robots with improved mobility, steerability, and controllability. However, challenges still exist in integrating the capabilities of macromolecular drug carrying, adaptive degradation, effective locomotion, and reliable targeted insertion in harsh GI tract environments. Further difficulties in biodegradable and drivable material synthesis, highly compacted multifunctional structure design, and intestinal mucosal barriers penetration also hamper robot-based targeted drug delivery in the GI tract.

### SUMMARY

In an example embodiment, the present disclosure provides a drug administering device, comprising a spherical body, a magnetic actuator arranged within the spherical body and configured to respond to an externally applied magnetic field, and a plurality of microneedles arranged on the spherical body, the microneedles being configured to penetrate tissue.

In a further example embodiment, the plurality of microneedles of the drug administering device each further comprise an active pharmaceutical ingredient (API) layer and a microneedle shell arranged to cover a radially outer periphery of the API layer.

In a further example embodiment, the plurality of microneedles of the drug administering device each further comprise a hyaluronic acid methacrylate (HAMA) hydrogel layer and a polyvinyl alcohol (PVA) layer. The HAMA hydrogel layer is arranged between the API layer and the PVA layer.

In a further example embodiment, the microneedle shell of the drug administering device comprises copolymers of methyl acrylate, talc, and sodium citrate.

In a further example embodiment, the microneedle shell of the drug administering device is configured to dissolve in an alkaline environment and thereby release the API layer.

In a further example embodiment, the microneedle shell of the drug administering device has a height of 200 μm to 800 μm and a penetrating tip pointing radially away from the spherical body, the penetrating tip forming an internal angle of up to 27°.

In a further example embodiment, the drug administering device further comprises a protective coating configured to cover the spherical body and the microneedles, the protective coating being configured to dissolve upon exposure to a stomach acid.

In a further example embodiment, the protective coating of the drug administering device comprises a mixture of sugar, water, and corn syrup.

In a further example embodiment, the diameter of the drug administering device including the protective coating is from 6.7 mm to 7.9 mm.

In a further example embodiment, the magnetic actuator of the drug administering device comprises iron (II,III) oxide (Fe₃O₄) nanoparticles and gelatin.

In a further example embodiment, the entire drug administering device is biodegradable within 60 minutes when in an environment having a pH greater than 7.

In a further example embodiment, the present disclosure provides a method for forming a drug administering device, comprising forming a first hemisphere and a second hemisphere, each hemisphere having a plurality of microneedles protruding therefrom. Each hemisphere is formed by inserting and pressing an active pharmaceutical ingredient (API) powder at least partially into copolymers of methyl acrylate arranged in a mold. The method further comprises inserting a magnetic actuator in the first or second hemisphere and pressing the first and second hemisphere together such that the magnetic actuator is secured within a sphere formed by the pressed hemispheres, and coating the sphere with a fondant paste.

In a further example embodiment, forming each hemisphere further comprises applying a hyaluronic acid methacrylate (HAMA) hydrogel layer to the copolymers of methyl acrylate and API powder and exposing the HAMA hydrogel layer to ultraviolet (UV) waves.

In a further example embodiment, forming each hemisphere further comprises applying a polyvinyl alcohol (PVA) layer to the HAMA hydrogel layer.

In a further example embodiment, the method for forming a drug administering device further comprises forming the magnetic actuator with at least two axial arms having an angle of 120° therebetween.

In a further example embodiment, pressing the first and second hemisphere together further comprises forming a bonding lip along which each of the first hemisphere and the second hemisphere are bonded to one another, the bonding lip extending radially about a circumference of the sphere and having a thickness of at least 180 μm.

In a further example embodiment, the present disclosure provides a method for administering an active pharmaceutical ingredient (API) to a patient, the method comprising actuating translational and rotational movement of a spiny milli-ball (SMB) robot within the patient's gastrointestinal (GI tract) by moving and rotating a magnet arranged outside of the patient, and causing a microneedle of the SMB robot to penetrate through a surface of the patient's GI tract by magnetic attractive force imposed on the SMB robot by the magnet arranged outside of the patient.

In a further example embodiment, the method for administering an API to a patient further comprises coating the SMB robot with a protective coating to form an orally ingestible SMB robot, the protective coating configured to cover microneedles of the SMB robot such that the microneedles do not scratch soft tissue of the patient between the patient's mouth and GI tract.

In a further example embodiment, the method for administering an API to a patient further comprises waiting for a predetermined period of time before causing the microneedle of the SMB robot to penetrate through the surface of the patient's GI tract, the predetermined period of time corresponding to a time required for the protective coating to dissolve within the patient.

### BRIEF DESCRIPTION OF THE DRAWINGS

FIG. 1 illustrates an orally ingested SMB bot 106 in a GI tract 108. Specifically, frame 100 illustrates a first method for treating

FIGS. 2A-2B illustrate magnetization of an SMB bot;

FIGS. 3A-3B illustrate SMB bot microneedle penetration of tissue;

FIG. 4 illustrates a method for producing an ingestible SMB bot;

FIG. 5 illustrates microscopic images of an SMB bot;

FIGS. 6A-6C illustrate experimental magnetization, load, and force measurements of an SMB bot:

FIGS. 7A-7B illustrate experimental vertical displacement and locomotion speed measurements of an SMB bot;

FIGS. 8A-8D illustrate experimentally observed penetration depths of an SMB bot subjected to different magnetic force strengths and gradients;

FIG. 9 illustrates a microscopic image of a small intestine penetrated by an SMB bot;

FIGS. 10A-10B illustrate experimental force and insertion depth measurements of an SMB bot subjected to varying magnetic force strengths and/or gradients;

FIGS. 11A-11B illustrate experimental insulin content and drug accumulation measurements;

FIGS. 12A-12B illustrate experimental blood glucose level and blood glucose changes; and

FIG. 13 illustrates a flowchart of a method for API delivery.

### DETAILED DESCRIPTION

The present disclosure provides for effective oral administration methods that can take a broad range of biologic drugs and release them at certain regions controllably, thereby avoiding the previously described disadvantages of known therapeutic procedures. A spiny milli-ball robot (SMB bot) is disclosed which has improved biocompatibility, biodegradability, maneuverability, and controllable insertion and release functionalities, and which also provides for biological macromolecular loading. The SMB bot is able to deliver and release biological macromolecular drugs in the GI tract with improved locomotion adaptivity, pH-sensitive degradability, and ideal levels of cytotoxicity. The present disclosure thereby provides for improvements in a broad spectrum of biomedical treatment applications, such as macromolecular drug-disease medication, selected high-dose chemotherapy, local inflammation treatment by oral administration, and other applications. The present disclosure further provides a simple and low-cost approach for orally delivered therapies.

In an embodiment, an SMB bot has three layers from inside to outside, including a magnetic skeleton for actuation, a core-shell microneedle array for drug loading and intestinal mucosa insertion, and a sugar paste coating to protect soft tissue from scratching. Biodegradable materials, such as copolymers of methyl acrylate (including, for example, Eudragit® L100-55 compounds), are first prepared and poured onto a mold to form a hollow shell body. Active pharmaceutical ingredients (API) are loaded in solid form to the shell body via pressing, thereby forming a sharp and highly loaded structure on the shell body. The API, which can be in powder form, is then covered with Hyaluronic Acid Methacrylate (HAMA) hydrogel, which is subsequently covered with polyvinyl alcohol (PVA) in a layer-by-layer process. The mold can be held for at least 24 hours to dry under room temperature conditions, after which the shell body can be peeled off form the mold. A magnetic actuating skeleton comprising a mixture of gelatin and Fe₃O₄ nanoparticles is provided so that the magnetic actuating skeleton is biodegradable and non-toxic. The magnetic actuating skeleton can be put in the center of a hollow shell body. A demolded SMB bot is immersed into fondant paste, which can comprise a mixture of sugar, water, and corn syrup. The fondant paste is configured to prevent damage to the soft tissue of a patient who ingests the SMB bot, such as scratching the SMB bot is ingested orally and passes through the patient's esophagus. The SMB bot can have a diameter sufficiently small to make it comfortable for oral administration. For example, the SMB bot can have a diameter of approximately 6 mm or approximately 6.7 mm to 7.9 mm.

In a method according to an embodiment of the present disclosure, an SMB bot is orally administered to a patient or orally ingested by a patient. The SMB bot then travels through the patient's esophagus and stomach to the patient's intestines. As the SMB bot travels through the esophagus and stomach, its protective coating covers its spines or microneedles and prevents them from scratching the patient. Upon reaching the patient's intestines, the protective coating is dissolved by intestinal juices. The SMB bot is then cause to move via movement and rotation of an external magnetic field originating from outside of the patient and within proximity to the SMB bot, thereby causing a corresponding movement and rotation of a magnetic actuating skeleton within the SMB bot. The SMB bot can then be anchored in position by one or more microneedles by increasing the strength of the external magnetic field on the SMB bot, either by increasing the magnetic strength of the external magnet or by bringing the external magnet in closer proximity to the SMB bot. As a result, the microneedles of the SMB bot penetrate a tissue layer surface of the intestine and are held in position until they dissolve, thus releasing underlying API into the site of penetration. The SMB bot may be configured to administer a dose of API via a single penetration site, or may be configured to repeat any of the foregoing steps in order to administer API over more than one penetration site. The SMB bot is then allowed to completely dissolve within the patient's intestines.

To achieve remote and precise actuation of the SMB bot, an external magnetic field can be applied to the SMB bot for controlled robot driving, the magnetic field being generated by an electromagnetic coil or permanent magnet. Compared to other remote actuation methods, such a magnetic field provides improved penetration and compatibility, as the magnetic field can go through most biological tissues noninvasively and adapt to various synthetic materials without side effects. Considering the biomedical applications in an in-vivo environment, the magnetic field is thus one of the most ideal driving methods for SMB bot actuation.

Degradation of the SMB bot can be controlled by exposure of the SMB bot to an environment having certain pH levels. The SMB bot, can include pH-sensitive materials that will convert to low molecular weight compounds in a weakly alkali environment. For example, the SMB bot can include EL 100-55 compounds, talc, and/or sodium citrate, which are universal pH-sensitive materials in weak alkali environments. Inclusion of pH-sensitive materials ensures that the SMB bot can protect drug or API macromolecules from premature activation during digestion due to the typically strong acidic environment found in a patient's stomach, which the SMB bot must pass through to reach the patient's GI tract.

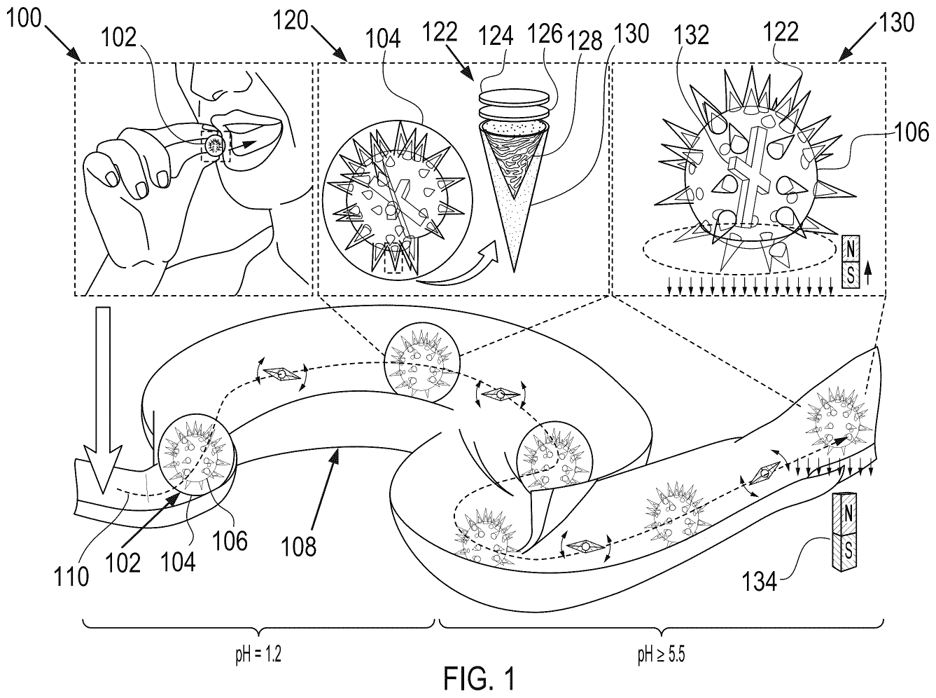

FIG. 1 illustrates an orally ingested SMB bot 106 in a GI tract 108. Frame 100 illustrates a first step in a treatment process in which an SMB bot 106 that is covered in a protective layer 104 to form an ingestible SMB bot 102 is orally ingested. The protective layer 104 coats the SMB bot 106 and its sharp tops or edges to protect a patient's soft tissue (e.g., the tissue in the esophagus) from scratching from ingestion until the ingestible SMB bot 102 has traveled to the GI tract 108. Once in the GI tract 108, which may have a pH of approximately 1.2 at stages closer to the patient's mouth, the ingestible SMB bot 102 begins traveling along a travel path 110 through the GI tract 108. As the ingestible SMB bot 102 travels through the GI tract 108 along the travel path 110, the protective layer 104 dissolves to expose spines 122 of the SMB bot 106. The protective layer 104 can include a fondant paste formed from, for example, a mixture of sugar, water, and corn syrup. The protective layer 104 can be configured to withstand a pH of 1.2 or to dissolve at a slow rate at lower pH values at or near 1.2 such that the spines 122 remain covered until later stages of the GI tract at which the pH increases. Later stages of the GI tract may have a pH of, for example, 5.5 or greater. At such elevated pH levels, the protective layer 104 is configured to dissolve to expose the spines 122.

The spines 122 of the SMB bot 106 are arranged about an outer periphery of the SMB bot 106, which can be spherically shaped for even spine distribution and to facilitate travel of the SMB bot 106 through the patient. As illustrated in frame 120, the spines 122 include, in order from a center of gravity of the SMB bot 106 to radially outward relative to the center of gravity of the SMB bot 106, a PVA layer 124, a HAMA hydrogel layer 126, an API powder layer 128, and a microneedle shell 130. The microneedle shell 130 can include, for example, an EL 100-55 compound. The microneedle shell 130 is configured to dissolve in a weak alkaline environment such as is found in the small intestine, thus exposing and releasing the API powder of the API powder layer 128 for administration directly to the GI tract 108.

As illustrated in frame 130, the SMB bot 106 also includes in its center a magnetic actuating skeleton 132. The magnetic actuating skeleton 132 is configured to be responsive to magnetic fields and fixed within the SMB bot 106 such that when the magnetic actuating skeleton 132 moves or rotates, the entirety of the SMB bot 106 moves or rotates with it. As a result, by controlled exposure of the SMB bot 106 to a magnetic field, the magnetic actuating skeleton 132 (and thus the entirety of the SMB bot 106) can be moved in a controlled manner through the GI tract 108 along a planned travel path 110.

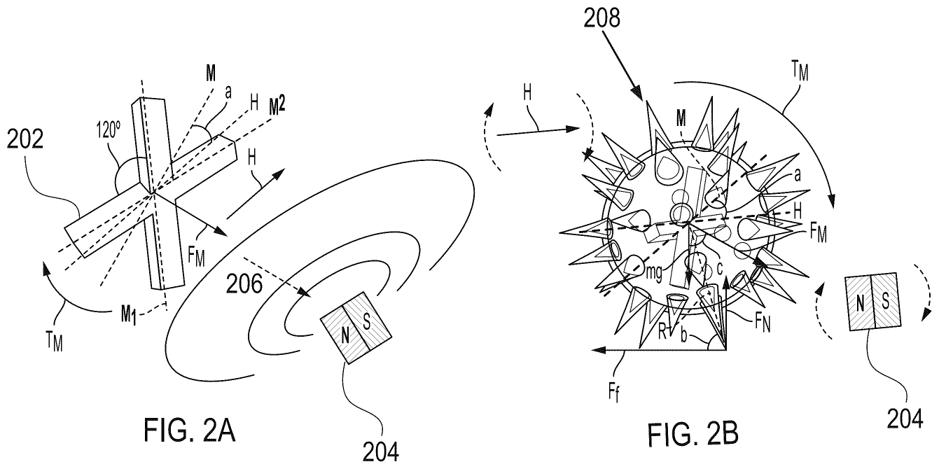

FIGS. 2A-2B illustrate magnetization of an SMB bot. A magnetic actuating skeleton 202 of an SMB bot 208 includes a first axis M₁ and a second axis M₂, each axis M₁, M₂ extending along an axial arm of the magnetic actuating skeleton 202. The angle between the first and second axis M₁, M₂ can be, for example, approximately 120 degrees. The magnetic actuating skeleton 202 is configured to include a magnetization M produced by magnetic materials within the magnetic actuating skeleton 202 at an angle a from a magnetic field direction H. As a result of the magnetization M, when the magnetic actuating skeleton 202 is exposed to an external magnetic field, the magnetic actuating skeleton is subjected to both a pulling force F_{M} and a torque T_{M}. The SMB bot 208 is also subject to a normal force FN opposing its weight mg and a friction force F_{f} opposing movement of the SMB bot 208 against a surface. The friction force F_{f} is at an angle b relative to an axis R extending from a center of gravity of the SMB bot 208 through the center of a spine experiencing the friction force F_{f}. The external magnetic field can be created by an external permanent magnet 204 or an electromagnet. In the case of an electromagnet, a magnetic field generation device that include three pairs of coils oriented perpendicular to each other can be used to create the external magnetic field, and electrical current through the coils can be controlled in order to affect the intensity and direction of the magnetic field generated. The external magnetic field also causes a magnetic gradient 206 in a direction between the magnetic actuating skeleton 202 and the external magnet 204. During exposure to the external magnetic field, the imposed torque T_{M} aligns the magnetic actuating skeleton's magnetization M with the magnetic field direction H, and a rolling motion can be achieved by continually rotating the magnetic field direction. The magnetic pulling force F_{M} can not only make positive work for the rolling motion, but also enhance the SMB bot's grip to avoid unexpected skidding. The external magnetic field may be altered by adjusting the positioning of the external magnet from which the external magnetic field is generated, for example by manual positioning, robotic positioning (e.g., via a robotic arm or a six-axis robot), or positioning via a three-dimensional motion platform.

The magnetic torque T_{M} and pulling force F_{M} are determined using relationships based on integration over volume of the magnetic actuating skeleton as follows:

$${\vec{T}}_{M} = {\int }_{V} \vec{M} \times \vec{B} ( x , y , z ) dV \qquad ( 1 )$$

$${\vec{F}}_{M} = {\int }_{V} ( \vec{M} \cdot \vec{∇} ) \cdot \vec{B} ( x , y , z ) dV \qquad ( 2 )$$

In equations (1) and (2), \vec{T}_{M} and \vec{F}_{M} are the torque and pulling force, respectively, acting on the SMB bot, \vec{M} is the magnetization of the magnetic actuating skeleton, \vec{B} is the magnetic induction intensity and ∇ is a divergence operator. Base on the foregoing, a dynamic model of the SMB bot can be achieved as:

$$J \ddot{θ} = {T}_{P} - {T}_{R} = {T}_{M} + {F}_{M} sin ( γ + β - \frac{π}{2} ) - mgR cos β \qquad ( 3 )$$

In equation (3), J and \ddot{θ} are the polar moment of inertia and the robot angular acceleration, respectively, of the SMB bot. Assuming a friction force can ensure no-slide motion, if the condition of positive torque T_{P} is larger than resistance torque T_{R} is met, controllable rolling of the SMB bot can be achieved.

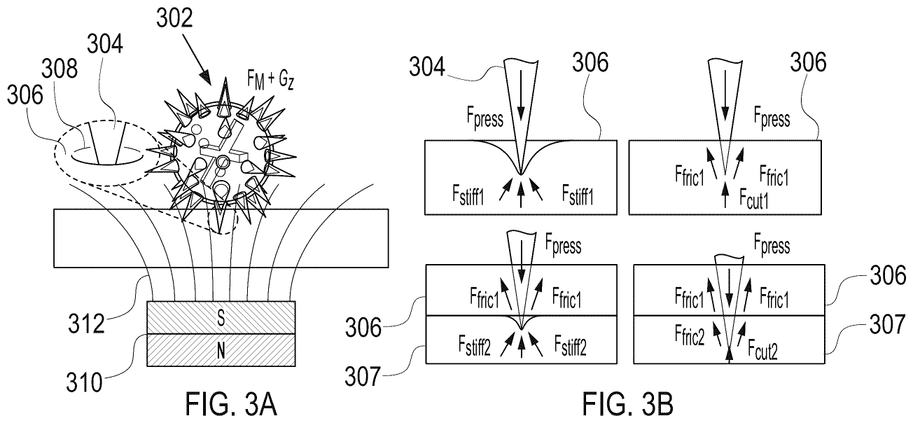

FIGS. 3A-3B illustrate SMB bot microneedle penetration of tissue. Specifically, FIG. 3A illustrates an SMB bot 302 having microneedles 304, at least one of which penetrates a surface of a first tissue layer 306 to form a deformation 308. An external magnet 310 with a magnetic field is arranged in proximity to the SMB bot 302 such that it is attracted to the external magnet 310. The imposed magnetic attraction in combination with gravity causes the microneedles 304 at the surface of the first tissue layer 306 to deform and penetrate the tissue, as illustrated in FIG. 3B.

The magnetic attraction of the SMB bot towards the magnet 310 can be changed by adjusting the strength and gradient of the magnetic field. When a permanent magnet is placed below an SMB bot with a distance d (i.e. xₚ=0, yₚ=0, zₚ=d), the total press force on the SMB bot can be expressed as:

$${F}_{P} = {\int }_{{V}_{R}} M \frac{\partial {B}_{z}}{\partial z} dV {\vert}_{z = d} + {G}_{z} \qquad ( 4 )$$

In equation (4), ∂B_{Z}/∂_{z} is the magnetic gradient along the z-axis and G_{Z} is the component of gravity along the z-axis. By comparing the intestinal wall's mechanical properties, the required puncture force and the corresponding magnetic field strength can be roughly determined. Theoretically, if the inequality F_{P}/S>E is met (wherein S is the contact area of the needle tip and E is the Young's modulus of the intestine), the intestine puncturing can be achieved by the SMB bot.

As illustrated in FIG. 3B, before insertion of a microneedle 304 into a tissue surface (which can be, for example, the mucosa of an intestine), the microneedle 304 is pushed against the surface of the first tissue layer 306 with a force Fₚᵣₑₛₛ that is opposed by forces resulting from the stiffness of the first tissue layer (F_{stiff1}). Once the microneedle 304 penetrates the surface of the first tissue layer 306 (e.g., when Fₚᵣₑₛₛ exceeds F_{stiff1}), the microneedle is opposed by frictional forces imposed by the tissue (F_{fric1}) and by the force required to cut the first tissue layer (F_{cut1}). The microneedle 304 then proceeds to penetrate the entirety of the first tissue layer 306 and deform a second tissue layer 307 (which can be, for example, the submucosa of an intestine). At this stage, when Fₚᵣₑₛₛ is opposed by F_{fric1} as well as forces resulting from the stiffness of the second tissue layer 307 (F_{stiff2}). Once the microneedle 304 penetrates the surface of the second tissue layer 307 (e.g., when Fₚᵣₑₛₛ exceeds each frictional force F_{fric1} and each stiffness force F_{stiff2}), the microneedle is opposed by frictional forces imposed by the first and second tissue layers 306, 307 (F_{fric1}, F_{fric2}) and by the force required to cut the second tissue layer (F_{cut2}).

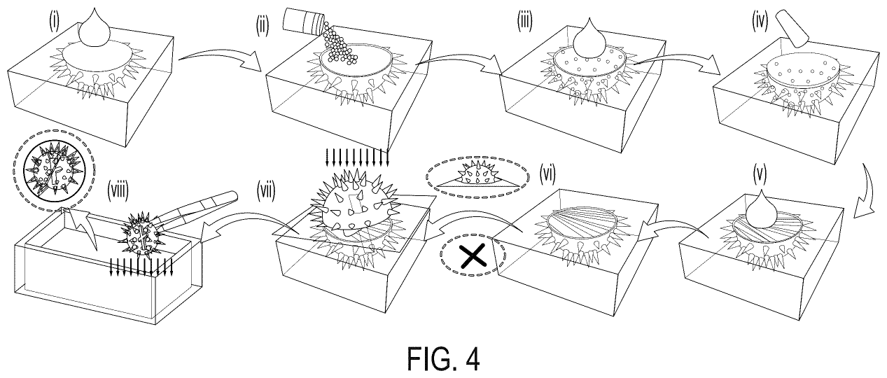

FIG. 4 illustrates a method for producing an ingestible SMB bot. The method includes a first step (i) in which a microneedle shell compound is prepared and inserted into a mold for creating half of a completed SMB bot. The microneedle shell compound can include EL 100-55 compounds and can be vacuumed and dried at a temperature of approximately 30° C. Next, in step (ii), API powders and/or drugs are pressed into the hollow portions of the microneedle shells formed in step (i). In step (iii), HAMA hydrogel is then arranged to cover and protect the API powders. The HAMA hydrogel can be spread over the entire exposed surface of the microneedle shell and API powders within the mold, thereby ensuring a full coating and coverage of all pressed API powders in an efficient manner. The HAMA hydrogel is allowed to dry before being exposed to ultraviolet (UV) waves in step (iv). Subsequently, in step (v), a binder, which can include PVA 20%, is added and allowed to dry. Next, in step (vi), the dried half-mold of the SMB bot is peeled from the mold. In step (vii), steps (i) to (vi) are then repeated to create a second half of the SMB robot into which a magnetic actuating skeleton is partially inserted, with the previously formed and peeled half being arranged over the exposed portion of the magnetic actuating skeleton and pressed onto the newly created half (for example, via tweezers configured to clamp the halves together from opposing ends) to create a whole. In step (viii), the whole SMB bot is then inserted into a sugar paste to add a protective layer over the entire SMB bot, thereby forming an ingestible SMB bot.

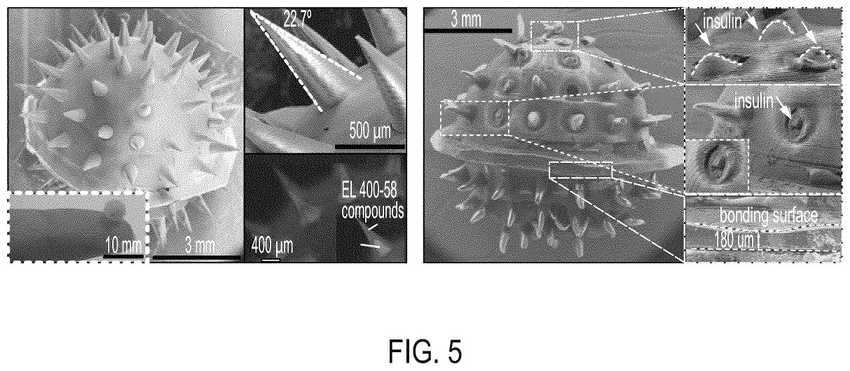

FIG. 5 illustrates microscopic images of an SMB bot. Each microneedle of the SMB bot can have a height of approximately 600 μm, a base diameter of approximately 300 μm, a core height (e.g., a height of the API powder core within the microneedle) of 400 μm, and a sharp corner forming an angle of 22.7°. A bonding surface having a thickness of approximately 180 μm is formed from molded halves being pressed together to form a lip about a circumference of the SMB bot. Through addition of macromolecule fluorescent drugs or insulin powder in the core of microneedle shells, a fluorescence microscopy and Scanning Electron Microscopy characterization, such as that illustrated in FIG. 5, can be carried out. Although the illustrated embodiment depicts microneedles having a height of approximately 600 μm. It will be readily appreciated that variation in the precise dimensions can be accommodated without departing from the spirit of the present disclosure. For example, the SMB bot's microneedles can have a length of from 200 to 800 μm.

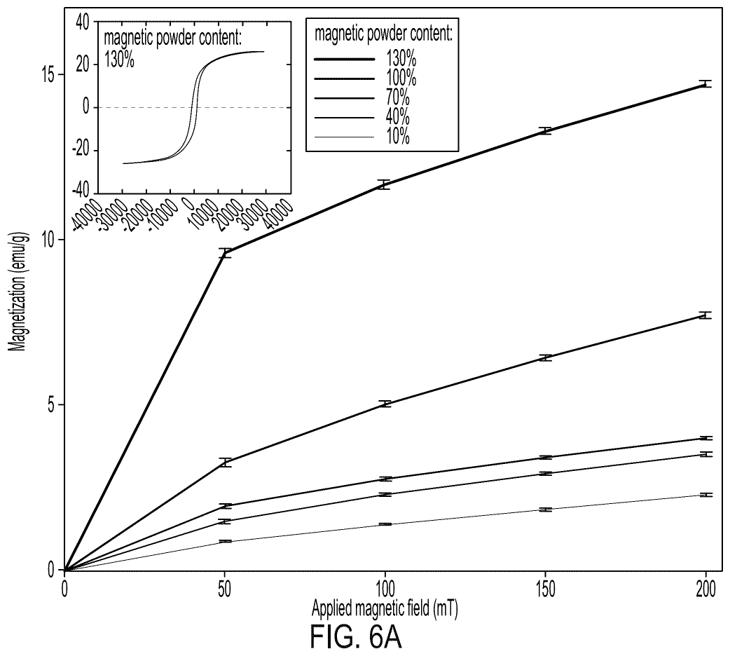

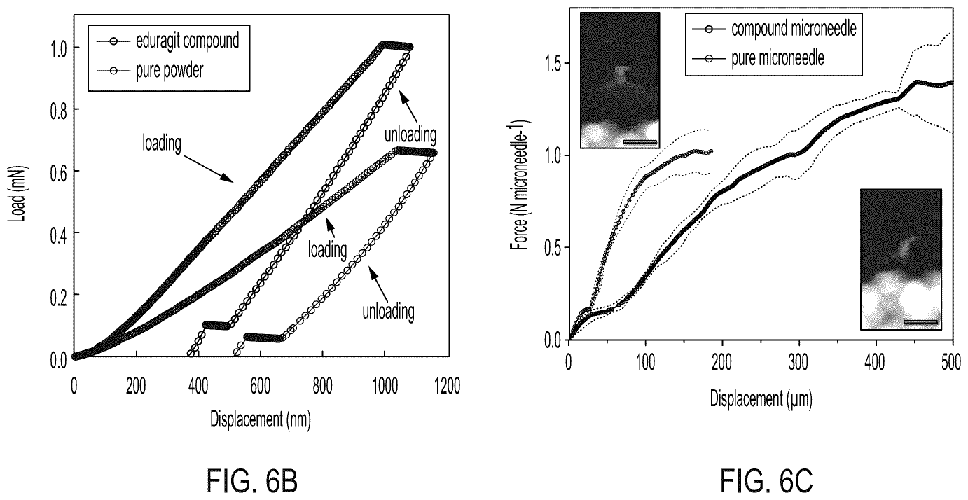

FIGS. 6A-6C illustrate experimental magnetization, load, and force measurements of an SMB bot. To guarantee the robustness of a manufactured SMB bot, mechanical performance of the microneedle core and shell structure and the magnetic actuating skeleton should be evaluated. For robotic locomotion, externally applied magnetic strength will affect the reliability of the SMB bot during its locomotion. Therefore, the magnetic actuating skeleton's magnetic strength can be tested under different ratios of gelatin and Fe₃O₄ particles that meet mechanical strength and motion flexibility requirements. In addition, mechanical performance of microneedle core and shell structures, including indentation hardness and penetration force, can be evaluated.

As illustrated in FIG. 6A, the magnetic actuating skeleton's magnetic moment density is characterized by different magnetic field strengths ranging from 0 to 200 mT, and the magnetization of the magnetic actuating skeleton increases as the magnetic powder content of the magnetic actuating skeleton increases. When the magnetic actuating skeleton's magnetic powder content is at approximately 130%, the magnetization of the skeleton's leg rises from 0 to approximately 14.9 emu/g with increases in applied magnetic field strength. This positive correlation between magnetization and magnetic field strength makes stable and controllable actuation of an SMB bot under an applied magnetic field possible.

To ensure that the SMB bot can be inserted into the mucosa of the small intestine, the materials and morphology of the SMB bot's microneedles need to be characterized. As such, the hardness of a microneedle's materials must be measured. As illustrated in FIG. 6B, unsynthesized copolymers of methyl acrylate are used as a blank control group. The average indentation hardness of the composite copolymers of methyl acrylate used in SMB bots according to the present disclosure is approximately 37 MPa, which is higher than the unsynthesized hardness of approximately 30 MPa. Meanwhile, the average indentation hardness of the selected composites is higher than several times to several hundred times that of various tissues and epidermis, meaning that the composite utilized according to the present disclosure is also hard enough for puncturing tissue and epidermis.

An SMB according to the present disclosure can be strong enough to penetrate tissue under compression without fracturing. For this purpose, compressive force tests were conducted to evaluate the mechanical strength of microneedles. As illustrated in FIG. 6C, compressive forces of synthesized microneedles is higher than that of unsynthesized microneedles. SMB bots with synthesized microneedle shells can tolerate about 1.25 N per microneedle, indicating successful tissue penetration can occur.

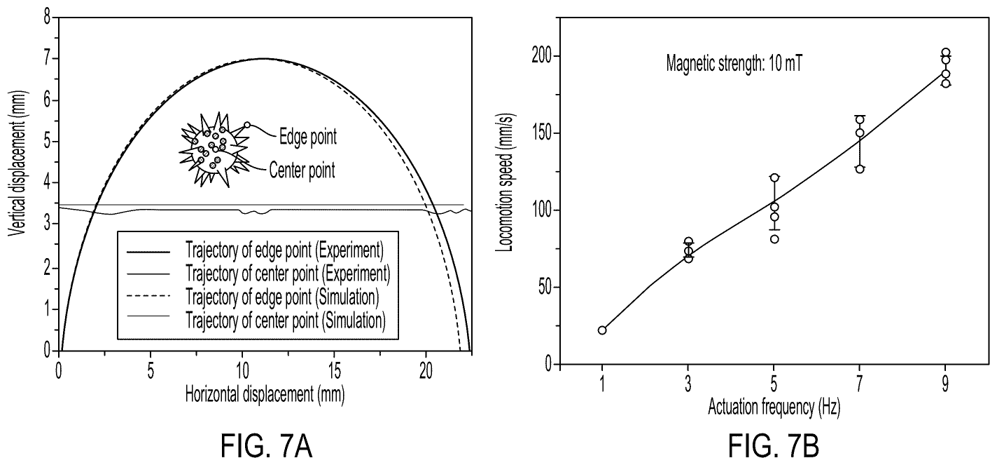

FIGS. 7A-7B illustrate experimental vertical displacement and locomotion speed measurements of an SMB bot. Specifically, FIG. 7A illustrates a dynamic model of an SMB bot with vertical displacement plotted against horizontal displacement. FIG. 7B illustrates a plot of experimental locomotion speed against actuation frequency for an SMB bot.

To verify controllable locomotion ability of the SMB bot, its rolling motion on a flat surface can first be tested. Then, the dynamic characteristics of the SMB bot can be evaluated by analyzing its motion trajectory, the hysteresis of rotation angle to the magnetic field, and the relationship between its motion speed and actuation frequency to optimize actuation and control of the SMB bot. The locomotion model of the SMB bot according to embodiments of the present disclosure has been tested on a flat surface. According to an analysis of the locomotion model, the trajectory of the SMB bot simulated by a theoretical model is consistent with that observed in actual experiment, verifying the support and reliability model as illustrated in FIG. 7B.

To further verify the locomotion adaptivity to a harsh bio-environment, the SMB bot's locomotion on flat areas and crease areas in the stomach and intestine can be tested using rugged, wet, acid, and viscous mucus layers as similar to an ex-vivo environment. The locomotion performance, including the rolling speed, obstacle overcrossing ability and fluctuation amplitude can be recorded for evaluation. Meanwhile, the structural stability of SMB bot under the biological environment (e.g., whether the microneedles or body structure of the SMB bot will be degraded during locomotion) can be recorded in real time and analyzed. Besides the theoretical analysis, comparative experiments to evaluate the practical motion performance of microswimmers, including its velocity and locomotion adaptability on different environmental surfaces and steering controllability, can be conducted. The velocity of three types of microswimmers under a 10 mT magnetic field with a rotating frequency from 1 Hz to 9 Hz can first be measured. As shown in FIG. 4C, the velocity of the SMB bot first rises as the frequency of the magnetic field increases.

The mobility of the SMB bot and corresponding locomotion characteristics are further demonstrable on different surface and an ex-vivo pig stomach. To clearly demonstrate the locomotion, three surfaces can be divided according to different roughness, i.e., a rough sandy surface, holes with a slightly rough surface, and a smooth glass surface. Under the trigger of external magnetic guidance having a magnetic strength of approximately 100 mT and a frequency of approximately 1 Hz with a magnet arranged about 5 cm beneath, an SMB bot according to embodiments of the present disclosure can move a certain distance. The movement of the SMB bot over a period of one second is affected by the roughness of the surface. The smoother the surface, the more efficient the movement of the robot, and the rougher the surface, the slower the robot moves. Experimental results verify that the SMB bot has excellent motion adaptability on both rough and smooth surfaces.

However, an actual in-vivo environment is filled with viscous digestive juices. As such, to demonstrate locomotion adaptivity in a harsh bio-environment, the SMB bot's dynamic locomotion was regulated remotely through a permanent magnet to experimentally carry out locomotion under continuous inverted-pendulum type on a real pig stomach. The pig stomach surface was covered with a mucus layer (pH 5 and viscosity 400 m·pas). Under the trigger of external magnetic guidance having a magnetic strength of approximately 100 mT, a magnetic gradient of approximately 1,500 mT/m, and a frequency of approximately 3 Hz, an SMB bot can move approximately 65 mm in approximately 12 seconds through the real pig stomach simulating a real gastrointestinal environment. The SMB bot thus advantageously exhibits untethered, efficient and controllable locomotion in harsh environments for various biomedical applications.

Continuous inverted-pendulum type locomotion includes locomotion closely resembling the inverted pendulum locomotion that occurs when humans walk. That is, the microneedles of an SMB bot can be approximated by the legs of a human in a locomotion model. In a human, as a supporting foot transitions to a swing phase from a position of rest, the human's center of mass (COM) shifts from directly above both feet to a position above and between the feet. Then, the COM shifts back to a position directly above both feet upon continued locomotion to another supporting phase in which both feet are essentially vertically aligned. Likewise, during a supporting phase, an SMB bot is positioned such that its COM is above two microneedles. As the SMB bot transitions to a swing phase, the COM shifts toward the supporting microneedle.

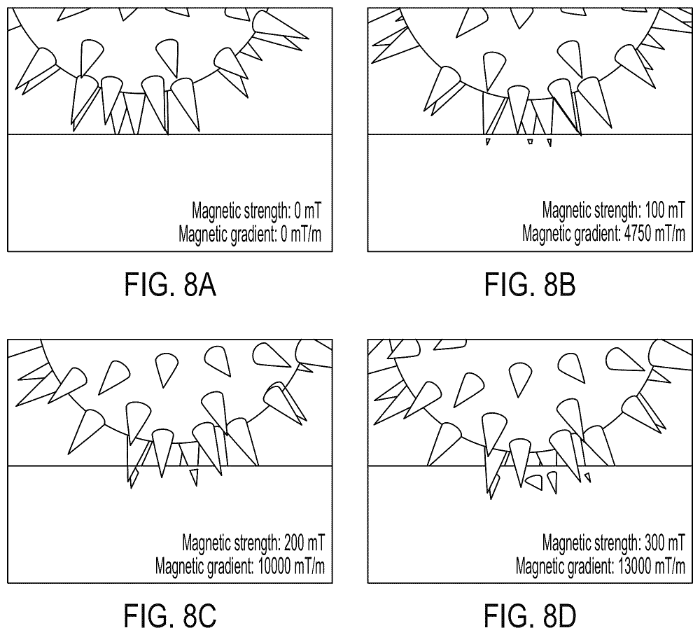

FIGS. 8A-8D illustrate experimentally observed penetration depths of an SMB bot subjected to different magnetic force strengths and gradients. FIG. 8A illustrates an SMB bot subjected to a magnetic strength of 0 mT and a magnetic gradient of 0 mT/m in which minimal to no penetration was observed. FIG. 8B illustrates an SMB bot subjected to a magnetic strength of 100 mT and a magnetic gradient of 4,750 mT/m in which slight penetration was observed. FIG. 8C illustrates an SMB bot subjected to a magnetic strength of 200 mT and a magnetic gradient of 10,000 mT/m in which increased penetration of tissue-like agar was observed. FIG. 8D illustrates an SMB bot subjected to a magnetic strength of 300 mT and a magnetic gradient of 13,000 mT/m in which still further increased penetration of tissue-like agar was observed.

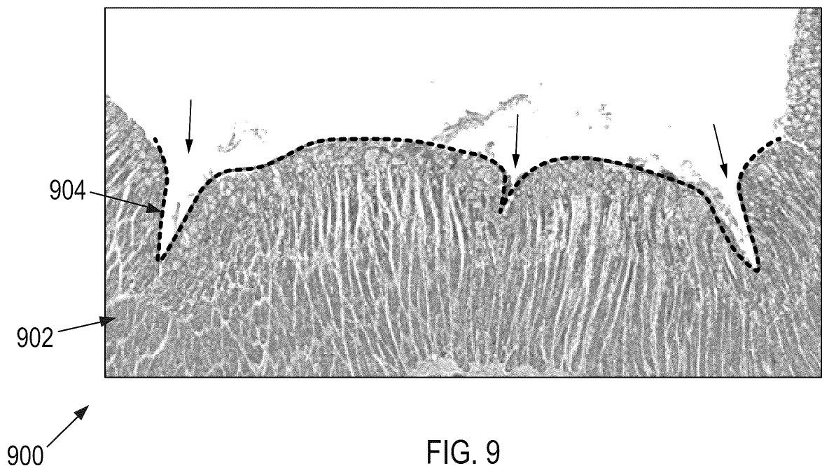

FIG. 9 illustrates a microscopic image of a small intestine penetrated by an SMB bot. Specifically, in an in-vivo study, an SMB bot according to embodiments of the present disclosure was placed in a capsule, infused orally into a rabbit's esophagus, and monitored through an endoscope. The capsule-wrapped SMB bot completely entered the small intestine of the rabbit and the remote SMB bot was inserted past the capsule's outer shell to the mucosa of the rabbit's small intestine using magnets. As the distance between the small intestine and the magnet decreased, the local magnetic field strength increased, and the SMB bot successfully pierced the intestinal mucosa in approximately 300 seconds. Microneedle traces in hematoxylin and eosin (H&E)-stained pictures of the penetrated tissue were obtained as additional proof of successful tissue penetration. After microneedle injection, pathological staining revealed intact tissue layers and no significant inflammatory responses. All penetration efficiencies were close to 100% and the penetration depths reached 500 μm in the small intestine tissue. FIG. 9 illustrates one such picture 900 carried out in the course of the experiment, and shows insertion points 904 into an H&E-stained tissue layer 902.

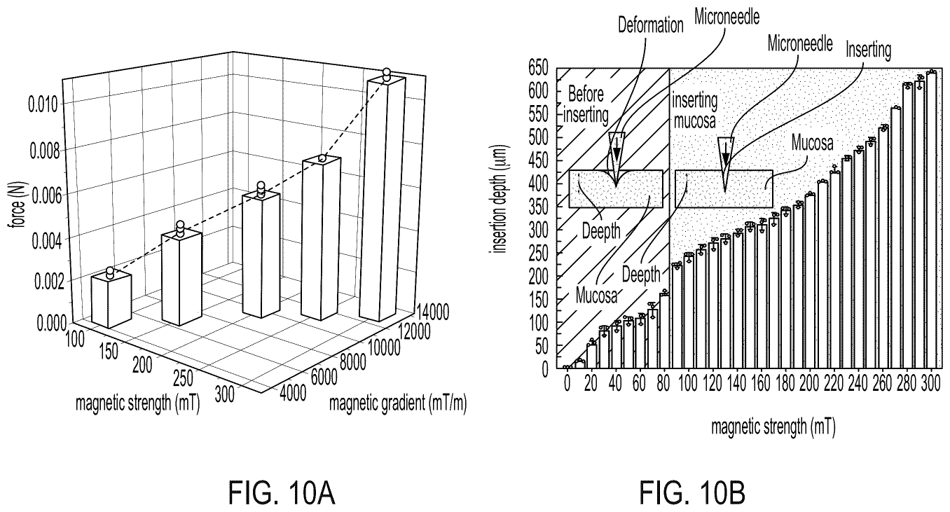

FIGS. 10A-10B illustrate experimental force and insertion depth measurements of an SMB bot subjected to varying magnetic force strengths and/or gradients. To verify the puncture ability of the microneedles of the SMB bot intuitively, an experiment was conducted to observe and record the insertion of SMB bot in tissue-like agar. Moreover, the generated press force under different magnetic fields was quantitatively detected and the relationship between applied press force and insertion depth was analyzed. An in-vivo animal experiment was conducted and an intestinal slice at a target area was analyzed, which served to further verify the effectiveness and safety of the punctures made by the SMB bot. Insertion of SMB bot microneedles into tissue-like agar under different magnetic fields was carried out to initially verify puncture ability. With an increase of the magnetic gradient from 0 to 13,000 mT/m, magnetic press force and the insertion depth increases, as illustrated in FIGS. 10A and 10B.

Specifically, increasing magnetic strength and magnetic gradient together was predictably observed to result in increased pressing force per microneedle of the SMB bot, as illustrated in FIG. 10A, and an increase in magnetic strength was observed to increase insertion depth of a microneedle, with penetration of the surface of the tissue-like agar beginning between approximately 80 to 100 mT, as illustrated in FIG. 10B. As further illustrated in FIG. 10B, the tissue-like agar's surface will slightly flex or deform when the tip of a microneedle abuts and presses against it without piercing or penetrating the agar. The magnetic attraction must be made strong enough to exceed a threshold value to achieve piercing or penetration. In experimental analysis, the insertion depth of microneedles changed abruptly when the magnetic field strength increased from 80 mT to 100 mT. Although insertion depth changed somewhat gradually and consistently from 0 to 80 mT, at the abrupt point of penetration, from 80 mT to 100 mT, the insertion depth changed rapidly from approximately 150 μm to 230 μm. The foregoing findings indicate that in order for the magnetic field to draw the microneedles of an SMB bot into the tissue-like agar, a magnetic force of at least 100 mT is necessary. Increases in the magnetic strength (e.g., the x-axis of FIG. 10B) can be achieved, for example, by decreasing the distance between a fixed SMB bot microneedle and an external magnet.

In addition, for a quantitative understanding of the magnet-actuated penetration of SMB bots, the penetrating forces of microneedles into agar mimicking the hardness of the intestinal mucosa as well as magnetic forces generated by an approaching magnet were recorded. The penetrating force was found to be about 0.013 N per microneedle when the microneedle fully penetrated into the agar. As the externally arranged magnet gradually approached the SMB bot during experimental observation, the generated magnetic force experienced exponential growth and reached more than 300 mT.

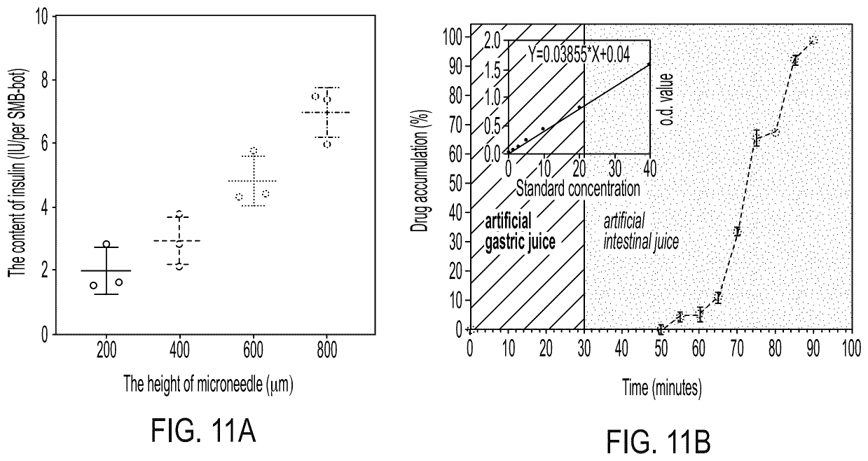

FIGS. 11A-11B illustrate experimental insulin content and drug accumulation measurements.

To experimentally analyze the intriguing pH-sensitive biodegradability and drug release capacity of the SMB bot according to embodiments of the present disclosure, the functional performance of a composite medicated SMB bot was estimated by adding to it approximately 3 to 5 kDa of fluorescein isothiocyanate-dextran (FITC-dextran) and Rhodamine B. The SMB bot was placed in an environment that mimicked the hardness and pH of human soft tissue by exposure to phosphate buffered salina (PBS), which has a weakly alkaline pH of approximately 7.1 to 7.2. The SMB bot was also placed on tissue-like agar. The degradation process of the SMB bot's core-shell microneedle was recorded at a macroscopic dimensional scale through microscopic bright and fluorescent images. This experimental analysis showed that the degradation of the SMB bot shell would be completed within 30 minutes, and then the core drug and/or API wrapped by the microneedles would be released at around 30 minutes from exposure to the weakly alkaline environment. The entirety of the experimentally observed SMB bot achieved complete degradation within 60 minutes at a pH greater than 7. Experimental results confirm that the entirety of the SMB bot remains stable in an acidic environment with a pH less than or equal to 5, which verifies the SMB bot's ability to protect active ingredients when traversing through a patient's stomach. The microneedle shell of the SMB bot dissolves and releases drugs and/or API when exposed to a pH of greater than or equal to 5.5, and the whole microneedle further dissolves and releases drugs and/or API more rapidly when exposed to a pH of 7 or greater.

To preliminarily evaluate the in-vitro drug delivery performance of the SMB bot, the SMB bot was loaded with insulin in a further experiment. As illustrated in FIG. 11A, when the SMB bot's microneedles were configured to be 600 μm in height, the SMB bot could contain a total of approximately 4.85 International Units (IU) of insulin. By increasing or decreasing the height of microneedles of the SMB bot, the insulin content can be adjusted, thus reducing drug waste while providing room for customization and personalization according to the needs of patients, such as diabetes patients. As each microneedle's height increases, the SMB bot's total insulin capacity increases. For example, when the microneedle height is adjusted to approximately 800 μm, the insulin content of a single SMB bot can reach approximately 7 IU of insulin. In general, the dosage of insulin required for an adult is 0.2 HU per kilogram. Accordingly, for a human male weighing approximately 60 kg, whose required dosage may be approximately 12 IU of insulin, three SMB bots with 600 μm microneedles may be required for treatment. FIG. 11A further illustrates additional experimentally measured insulin content values based on varying SMB bot microneedle heights. It will be readily appreciated that the relationship between microneedle height and insulin content is not exclusive only to insulin, and applies similarly or equally to other drugs and/or APIs.

To further verify and evaluate the macromolecule drug release profiles of the SMB bot according to embodiments of the present disclosure in various pH buffer solutions, loaded drug dissolution tests of the SMB bot were carried out under various pH conditions. Results of the experiment are illustrated in FIG. 11B. Insulin was again used as a model drug in the SMB bot for this experiment. The SMB bot was dissolved in artificial gastric juice having a pH of approximately 1.2 and artificial intestinal juice having a pH of approximately 7.2 and analyzed using an enzyme-linked immunosorbent assay (ELISA) kit for building a standard equation using a calibration curve based on different pH buffer solutions. Then, prepared SMB bot samples were placed into a rotating basket containing different pH buffer solutions having a pH of approximately 1.2 and 7.2. The samples in the basket were measured every 10 minutes, and then the measured sample solutions were restored to their experimental container. To confirm consistency of the results of this experiment, the experiment was repeated three times using three different samples. The various drug detection wavelengths will show different sharp peaks in a spectrum. According to the relation between the standard equation and these peaks, the drug loading efficiency (LE) of a sample can be determined by the following equation:

$$LE ( \% ) = \frac{( drug weight measured )}{( drug weight calculated from the preparation )} * 100 \% \qquad ( 5 )$$

A further experiment was carried out for verification of the biological macromolecular therapy ability of the SMB bot according to embodiments of the present disclosure. For these further experiments, insulin-loaded SMB bots were delivered orally to a New Zealand rabbit's intestinal tissue via application of an external magnetic field. The blood glucose level of the rabbit was measured for verification of biological macromolecular therapy superiority. Two groups of the SMB bot were designed to verify the SMB bot's performance in terms of drug carrying and releasing for adjusting blood glucose levels. In a first group, SMB bots were encapsulated with insulin and subjected to full degradation and drug release on the surface of the rabbit's intestine. In a second group, SMB bots were encapsulated with insulin and inserted into the tissue by applying a magnetic field (and thus a magnetic force) on the SMB bot, and also subjected to full degradation. During six hours of intermittent blood glucose observation, the changing blood glucose levels were observed and recorded to evaluate and confirm the effectiveness and advantages of the biological macromolecular therapy using an SMB bot.

In addition, an in-vivo pharmacokinetic analysis experiment was designed to further verify the biological macromolecular therapy. Here, the SMB bot is orally fed into the rabbit's esophagus and the position of the SMB bot is observed in real-time via X-rays. When the robot arrives at the intestine, an external magnetic field was applied to attract it for insertion. Then, 1 mL of whole blood was collected and extracted to affirm insulin concentration every half hour for six hours. The collected blood values were analyzed and compared with the standard insulin half-life, by which the effectiveness and advantages of the biological macromolecular therapy were evaluated and confirmed.

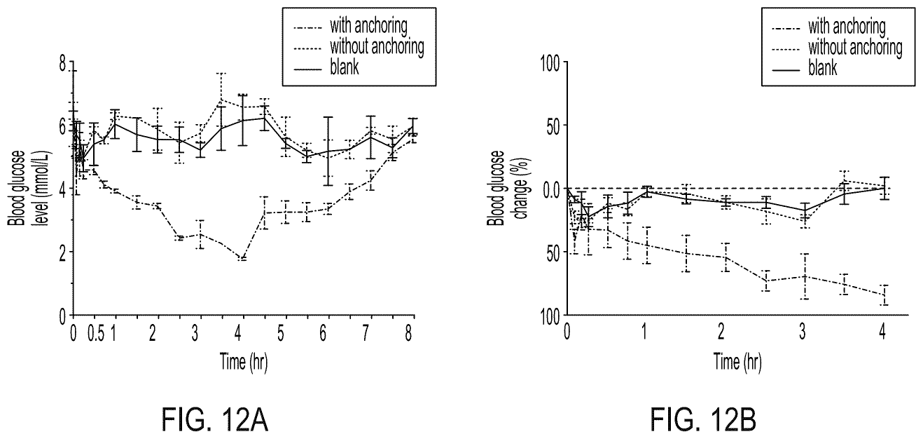

FIGS. 12A-12B illustrate experimental blood glucose level and blood glucose changes based on a further experimental analysis. A further experiment was conducted using a simple control macromolecule drug (insulin) release on New Zealand rabbits that were 8-12 weeks old and between 1.8 to 2.1 kg. The results, as illustrated in FIGS. 12A and 12B, showed that blood glucose levels decreased after the SMB bot was anchored on the intestine, verifying the SMB bot's ability to transport and deliver macromolecular drugs. The achieved anchoring was sufficient to overcome the peristalsis of the rabbit's GI tract. This was accomplished by using a capsule to send SMB bots holding 20 IU of insulin directly to the small intestine, where the intestinal mucosa was penetrated due to forces imposed by an external magnet. Glucose levels in the treated rabbit were observed to return to normal levels within 8 hours.

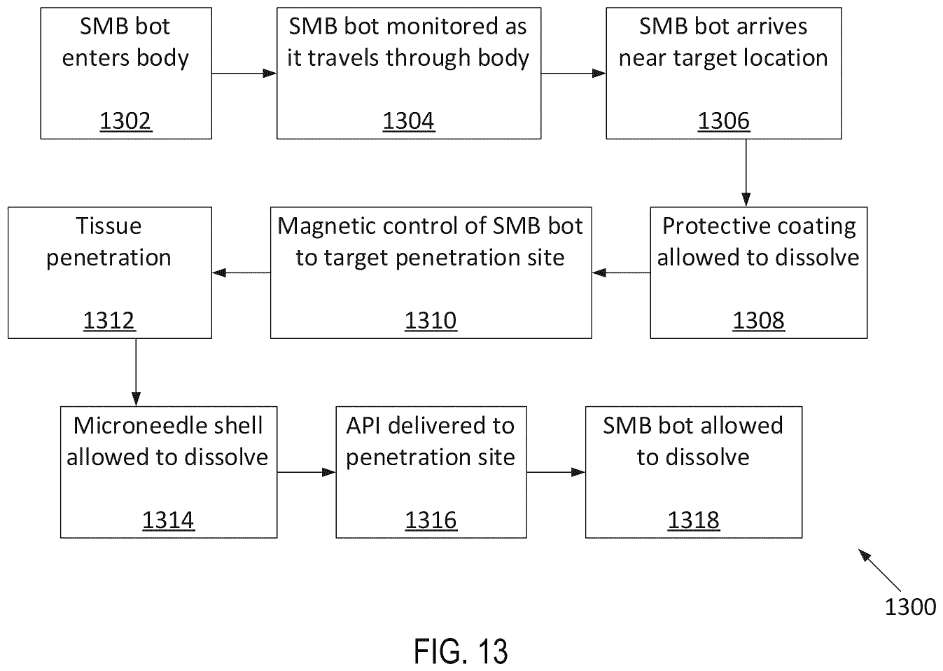

FIG. 13 illustrates a method 1300 for SMB bot API delivery. In a first step 1302, the SMB bot enters the body of a patient (human or animal) to which the API is meant to be administered. The SMB bot may enter the body by the patient by the patient orally ingesting the SMB bot or by the SMB bot being manually administered or delivered into the GI tract of the patient. In a subsequent step 1304, the SMB bot is monitored as it travels through the body of the patient. The SMB bot can be monitored via an imaging device. The imaging device can include, for example, ultrasound and/or digital radiography. The SMB bot then arrives near a target location in step 1306, and the protective coating of the SMB bot is allowed to dissolve in step 1308 to expose the SMB bot's microneedles. In a further step 1310, the SMB bot is controlled via a magnetic field created by a device or a magnet arranged external to the patient's body. As described above in the present disclosure, the magnetic field may be created by a permanent magnet and/or an electromagnet, and the magnetic field may be manipulated manually or automatically controlled via current adjustment and/or positional changes in a manner configured to cause the SMB bot to move (e.g., via inverted-pendulum type locomotion) to a more precise target location. Once the SMB bot is at a target penetration site, tissue penetration is initiated in step 1312 by increasing the strength of the external magnetic field, which causes the one or more of the SMB bot's microneedles to penetrate the tissue layer at the target penetration site. The shell of the one or more microneedles is then allowed to dissolve by waiting for a waiting period in step 1314. Once the microneedle shell(s) dissolve, API layer(s) previously covered by the microneedle shell(s) are exposed in step 1316, which allows the API to disperse from the SMB bot and be delivered into the penetration site due to their close physical proximity to the penetration site and exposed tissue therein. In a final step 1318, the SMB is allowed to dissolve under influence of the GI tract environment, leaving non-toxic API and dissolved byproducts of the SMB bot to be pass through the patient without further significant effect on the patient.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A drug administering device, comprising:
a spherical body;
a magnetic actuator arranged within the spherical body and configured to respond to an externally applied magnetic field; and
a plurality of microneedles arranged on the spherical body, the microneedles being configured to penetrate tissue.

2. The drug administering device of claim 1, wherein the plurality of microneedles each comprise:
an active pharmaceutical ingredient (API) layer; and
a microneedle shell arranged to cover a radially outer periphery of the API layer.

3. The drug administering device of claim 2, wherein the plurality of microneedles each further comprise:
a hyaluronic acid methacrylate (HAMA) hydrogel layer; and
a polyvinyl alcohol (PVA) layer,
wherein the HAMA hydrogel layer is arranged between the API layer and the PVA layer.

4. The drug administering device of claim 2, wherein the microneedle shell comprises copolymers of methyl acrylate, talc, and sodium citrate.

5. The drug administering device of claim 2, wherein the microneedle shell is configured to dissolve in an alkaline environment and thereby release the API layer.

6. The drug administering device of claim 2, wherein the microneedle shell has a height of 200 μm to 800 μm and a penetrating tip pointing radially away from the spherical body, the penetrating tip forming an internal angle of up to 27°.

7. The drug administering device of claim 1, further comprising a protective coating configured to cover the spherical body and the microneedles, and wherein the protective coating is configured to dissolve upon exposure to a stomach acid.

8. The drug administering device of claim 7, wherein the protective coating comprises a mixture of sugar, water, and corn syrup.

9. The drug administering device of claim 7, wherein the diameter of the drug administering device including the protective coating is from 6.7 mm to 7.9 mm.

10. The drug administering device of claim 1, wherein the magnetic actuator comprises iron(II,III) oxide (Fe₃O₄) nanoparticles and gelatin.

11. The drug administering device of claim 1, wherein the entire drug administering device is biodegradable within 60 minutes when in an environment having a pH greater than 7.

12. A method for forming a drug administering device, comprising:
forming a first hemisphere and a second hemisphere, each hemisphere having a plurality of microneedles protruding therefrom, and each hemisphere being formed by inserting and pressing an active pharmaceutical ingredient (API) powder at least partially into copolymers of methyl acrylate arranged in a mold; inserting a magnetic actuator in the first or second hemisphere and pressing the first and second hemisphere together such that the magnetic actuator is secured within a sphere formed by the pressed hemispheres; and
coating the sphere with a fondant paste.

13. The method of claim 12, wherein forming each hemisphere further comprises applying a hyaluronic acid methacrylate (HAMA) hydrogel layer to the copolymers of methyl acrylate and API powder and exposing the HAMA hydrogel layer to ultraviolet (UV) waves.

14. The method of claim 12, wherein forming each hemisphere further comprises applying a polyvinyl alcohol (PVA) layer to the HAMA hydrogel layer.

15. The method of claim 12, further comprising forming the magnetic actuator with at least two axial arms having an angle of 120° therebetween.

16. The method of claim 12, wherein pressing the first and second hemisphere together further comprises forming a bonding lip along which each of the first hemisphere and the second hemisphere are bonded to one another, the bonding lip extending radially about a circumference of the sphere and having a thickness of at least 180 μm.

17. A method for administering an active pharmaceutical ingredient (API) to a patient, the method comprising:
actuating translational and rotational movement of a spiny milli-ball (SMB) robot within the patient's gastrointestinal (GI tract) by moving and rotating a magnet arranged outside of the patient; and
causing a microneedle of the SMB robot to penetrate through a surface of the patient's GI tract by increasing a magnetic attractive force imposed on the SMB robot by the magnet arranged outside of the patient.

18. The method of claim 17, further comprising coating the SMB robot with a protective coating to form an orally ingestible SMB robot, the protective coating configured to cover microneedles of the SMB robot such that the microneedles do not scratch soft tissue of the patient between the patient's mouth and GI tract.

19. The method of claim 18, further comprising waiting for a predetermined period of time before causing the microneedle of the SMB robot to penetrate through the surface of the patient's GI tract, the predetermined period of time corresponding to a time required for the protective coating to dissolve within the patient.
